(19) United States
(12) Patent Application Publication
(10) Pub. No.: US 2015/0051700 A1
(43) Pub. Date: Feb. 19, 2015

(54) SYSTEM FOR ATTACHING A LIGAMENT GRAFT

(71) Applicant: BREVEXCO SPRL, Bruxelles (BE)

(72) Inventor: Michel Collette, Bruxelles (BE)

(73) Assignee: BREVEXCO SPRL, Bruxelles (BE)

(21) Appl. No.: 14/387,468

(22) PCT Filed: Mar. 22, 2013
(86) PCT No.: PCT/EP2013/056163

§ 371 Date: Sep. 23, 2014

(30) Foreign Application Priority Data
Mar. 23, 2012 (BE) 2012/0204

(51) Int. Cl. A61F 2/08

(52) U.S. Cl. CPC A61F 2/0811; A61F 2002/0858; A61F 2002/0852
USPC 623/13.14

(57) ABSTRACT

A system including a pin and a wire suitable for fixing a ligament graft to a bone, consists a body having a cylindrical cavity capable of receiving a wire itself attached to the end of the ligament graft and intended to be threaded into a tunnel formed in the bone, the cavity traversing the pin from side to side, through the centre of same, forming an entry port and an exit port of the wire, the body being capable of blocking the wire and being positioned in a housing formed in the cortical portion of the tunnel in the bone. The wire is blocked in the pin preferably using a sleeve-cap compressing the cylinder from the outside to the inside.

This invention relates to a new system for fixation of a ligament graft that can be applied more particularly at the knee, as well as pins, and other fixation means especially suited to this system.

The tearing of the anterior cruciate ligament (ACL) of the knee constitutes the most frequent traumatic ligament injury in sports pathology (about 200,000 cases per year in the United States). When it occurs, such an injury seriously compromises the physical abilities of the young athlete and can also, even with more sedentary subjects, cause an intermittent and sometimes very hindering functional impairment (repeated buckling, recurrent sprains, etc.).

### BACKGROUND OF THE INVENTION

For several decades, various surgical techniques have been used to give the traumatized knee a functional capacity as close as possible to the norm.

Because of the inconsistency of their results, the repair techniques by simple suture have gradually been supplanted by techniques for replacing the torn ligament by supplying fibrous tissue (ligament grafts) taken from the patient himself (autografts) or from clinically dead donors (allografts).

Two major types of grafts are competing for first place in terms of frequency of use.

The technique that is the oldest and still very widespread in the world is known in France by the technical name of “Kenneth-Jones.” It consists of taking an 8-9 cm portion of patellar tendon (which extends from the lower pole of the patella to the anterior tibial tuberosity) while preserving the bone insertion of each end of the tendon strip by means of a small bone block removed at the expense of the patella and of the anterior tibial tuberosity, intended to facilitate the fixation of the graft in the knee of the recipient. After having made two bone tunnels (one in the external femoral condyle and the other in the tibia) located in the extension of the anatomical insertion sites of the torn ligament, the graft is introduced into the knee, and its positioning is adjusted in such a way that the tendon part of the graft lines up with the intra-articular path of the crossed ligament and in such a way that the bone blocks are buried in the bone tunnels, femoral and tibial, or they will be fixed, most often by tightening, by means of a so-called interference screw, introduced between the bone block of the graft and the wall of the bone tunnel. This technique gives generally satisfactory results from a mechanical standpoint but is not devoid of risks. Most frequently, residual, sometimes permanent, pain persists in the middle of the anterior surface of the knee, at the site of the harvesting of the graft. Other complications, such as a secondary tearing of the weakened patellar tendon or even a fracture of the patella, have also been described, prompting a growing number of surgeons to turn to other, less invasive techniques.

The second major technique for reconstruction of the ACL makes use of tendon structures belonging to the group of “hamstring” muscles (extending from the ischium to the proximal tibia) and in particular to the gracilis and semitendinosus, located on the posterior face of the thigh and ending on the inner face of the proximal tibia. The gracilis/semitendinosus grafts have several advantages. It is possible to harvest these tendons from a minimum cutaneous incision of 2 to 3 cm maximum without the least risk of chronic residual pain. The secondary loss of strength at harvesting is less than after harvesting from the patellar tendon. Each tendon generally has a length of 25 to 30 cm, which makes it possible by folding them onto themselves to make a graft composed of 4 tendon strands of 12 to 15 cm. The intrinsic strength of these 4 tendon strands (300 to 400 daN) in principle very greatly exceeds the strength of the natural ACL (180 to 200 daN) but in reality, the actual tear strength of the graft depends on the strength of the weakest link of the fixation-graft-fixation chain.

Now the fixation of the gracilis/semitendinosus grafts is relatively problematic because of the absence of bone blocks as they exist in patellar tendon grafts. The multiplicity of means for fixation of gracilis/semitendinosus grafts perfectly illustrates the difficulty of obtaining a strong anchoring in a reproducible way. The types of fixation can be classified according to the level where the fixation is made: either at the intra-articular opening of the bone tunnel (corresponding to the insertion site of the ACL), or in the intra-bone segment of the tunnel, or at the extra-articular opening of the bone tunnel (cortical fixation).

Numerous experimental works have shown that the closer the fixation gets to the anatomical insertion site of the ACL, the more the mechanical behavior of the graft improves. Unfortunately, the tear strength of the fixations generally decreases as the anatomical insertion is drawn closer to and depends very much on the quality of the bone where the anchoring is made. The softer the bone, the weaker the tear strength. These fixations therefore do not make it possible to ensure the reproducibility of the anchoring strength, from one individual to the next. Not knowing the strength effectively obtained in each case, it is advisable to protect all those operated upon by means of canes or braces for several post-operative weeks. Conversely, the so-called cortical fixations, made at a distance from the anatomical insertion site of the ACL, obtain a very strong anchoring in a reproducible way. Unfortunately, the more the fixation-graft-fixation chain is lengthened, the more elastic it becomes, and this compromises the mechanical properties of the graft, as numerous experimental and clinical studies have shown.

In 2002, the applicant tested in a mechanical laboratory a new system called the TLS (Tape Locking Screw) system that made it possible to attach the graft near the anatomical insertion opening while implementing its anchoring in the vicinity of the cortical bone. This system was the object of patents (see PCT publication WO2004/045465 and WO2007/147634).

The graft consists of a single tendon, generally the semitendinosus, wound on itself so as to create a closed loop having 4 strands. The loop is fixed by one to two suture points penetrating the tendon strands at each end of the loop. Mechanical strength tests made on this type of graft have invariably demonstrated a tensile strength going from 100 to 200 daN, thus exceeding by far the actual needs in immediate post-operative phase (60-70 daN). The system for fixation of this graft is also original. Textile strips (polyethylene terephthalate) are passed freely through each pole of the ligament loop. They are then pulled through each bone tunnel from inside to outside and recovered on the outside of the knee, at the femur, and at the tibia. The introduction of the graft in the knee is obtained by simple pulling on the strips, and each end of the graft enters into its respective bone tunnel. A special screw, called a TLS screw, is then introduced from outside to inside in each bone tunnel. The band is then wedged between the blunt thread of the screw and the receiving bone, thus ensuring the fixation of the graft. The length of the screw (20-25 mm) makes it possible to counteract the sliding of the band practically until contact with the graft, thus avoiding the problems of elasticity pertaining to systems for suspension of the graft on a cortical fixation element (endobutton type). Strength tests performed in the laboratory have demonstrated in high-density bones values that can reach more than 220 daN. However, this strength remains highly dependent on the surrounding bone quality and can fall under the threshold of 50 daN in case of low bone density.

Although the TLS system has not ceased being used (more than 20,000 cases have been performed with this system since its launch in 2007) to the great satisfaction of its users, this invention proposes introducing a new ligament anchoring system derived from the TLS system, which keeps its advantages while avoiding its problems.

The making of a short graft configured in a closed loop with 4 strands constitutes one of the strong points of the TLS technique. The traditional preparation of gracilis/semitendinosus grafts necessitates the harvesting of 2 tendons that are then folded back on their center, which also produces a graft with 4 strands, but only the folded side is suitable for having some suspension element or other pass through there. On the other hand, the other pole consists of 4 free strands whose fixation remains to this day very problematic. Actually, these 4 strands have an unequal diameter and different length. It is technically difficult to maintain an equal tension on the 4 strands during the installing of the fixation. If the tension is unequal, this means that the crimping of the fibers in case of stress will be applied successively by beginning with the most stretched strand and finishing with the least stretched. In the TLS system, the tendon loop is closed, obtaining two loop handles that are available for pulling instead of one. The stressing of this type of loop creates tension forces that are automatically distributed equally in the 4 strands (pulley effect). In addition, this mechanically advantageous loop necessitates harvesting a single tendon instead of two. Finally, laboratory tests have clearly demonstrated that this type of assembly obtains a graft that is at once very strong and very rigid because of its reduced size.

However, in contrast with traditional systems that use long grafts of 12 to 15 cm, the size of a TLS-type closed loop does not exceed 50 to 60 mm, which inevitably calls for the usage of bridging elements intended to extend the graft at its two poles to ensure its fixation. This type of bridge is commonly used in surgery in the form of textile rings or strips that pass through the ligament loop handle on one part and are attached to a rigid element generally anchored on the cortical bone at the external opening of the bone tunnel. As we have already mentioned, this type of bridge obtains a strong but relatively elastic fixation because of the intrinsic flexibility of the bridging element located between the graft and the fixation. The TLS screw makes it possible to avoid this drawback since it enters into the bone tunnel itself and attaches the band practically until contact with the graft. The free, elastic portion of the suspension band is thus reduced to a negligible length, eliminating the problem of elasticity.
The quality of the fixation unfortunately remains dependent on several other factors:
   the quality of the bone where the anchoring takes place;
   the softer the bone, the more the band is susceptible to sliding when it is subjected to a pulling
   the quality of screwing;
   the fixation will be optimal if the screwing is optimal. In contrast, the strength of the fixation can be affected by screwing defects such as an excessive introduction of the screw that risks expelling the graft, conversely an introduction of the screw that is not deep enough diminishing the number of turns that wedge the band, or finally a divergence between the axis of the tunnel and the axis of introduction of the screw,
   the method of deploying the band in the bone tunnel;
   during laboratory tests, one makes sure that the exposure of the band to the interior walls of the bone tunnel is maximum, so as to optimize the fixation surface. In contrast, in reality, it is not possible to control and especially master the positioning of the band within the bone tunnel. It can be imagined that the band could twist during its introduction or else that the strips could have a tendancy to overlap within the tunnel, thus reducing the surface exposed to the turns of the screw.

### SUMMARY OF THE INVENTION

The invention proposes a pin system having two components that is intended to attach a suspension cord of a ligament graft previously introduced into a bone tunnel.

The first component consists of an oblong body, cylindrically- or egg-shaped, pierced with a cylindrical cavity able to receive a cord, itself fixed, via a loop handle, to an end of the ligament graft and intended to be threaded into a tunnel formed in the bone, said cavity passing through the first component from one side to the other, through its center, while forming an entry opening and an exit opening for the cord, said body being able to lock the cord in the bone tunnel. This first component can appear in the form of a pin that is able to be compressed toward the axis of the oblong body under the effect of an outside thrust that is perpendicular to the axis, and thus to immobilize the cord. The second component consists of a compression means, for example in the form of a cap-sleeve intended to be introduced between the interior wall of the bone tunnel and the external wall of the oblong body of the pin, so as to reduce the inside diameter of it and to cause the wedging of the cord in its center.

An alternative process for locking the cord is based on the ratchet effect and involves a self-locking pin with a suitable cord.

### PURPOSE AND DISCUSSION OF THE INVENTION IN RELATION TO THE PRIOR ART

This invention makes it possible to optimize the elasticity characteristics of the suspension system, to avoid problems of bone quality, of screwing quality, and of deployment of the band of the prior art.

The invention makes it possible, in addition, to be able first to perform, freely and independently, the adjustment of the position of the graft in the bone tunnel and second to add the fixation, which sets the graft in the position previously selected. This property of the invention thus distinguishes it very clearly from several anchoring systems that necessitate a prior joining of the graft to the anchor before implementing the fixation, which necessarily involves an obligatory movement of the graft, identical to the movement transmitted to the pin to obtain its fixation in the bone (see, for example: US 2007/0162022 A1, WO 2011/160166 A1).

In fact, the invention proposes a pin system with two components intended to wedge a suspension cord of a ligament graft as close as possible to the graft, so as to limit as much as possible the elasticity of the system, while taking advantage of the high strength of the cortical bone (at the exit of the bone tunnel, i.e., at a distance from the graft).

Several pin systems with two components intended for the fixation of a natural or artificial ligament graft are known.

For example, the patent U.S. Pat. No. 5,108,431 discloses a pin with two elements composed of a cone-shaped external sleeve introduced into the bone at the exit of the bone tunnel and through which the ligament to be attached passes. On this ligament, a second element slides that is also cone-shaped, radially deformable, which by fitting into the external sleeve wedges the ligament in its center. By the nature of its conical shape, such a device is necessarily applied at the exit of the bone tunnel, therefore at a distance from the graft, which can considerably increase the elasticity of the fixation-graft-fixation complex. In contrast, this system can only be used at one of the two ends of the bone tunnels. Actually, to obtain the tightening effect, the mobile sleeve must necessarily be moved in the reverse direction of the tensioning movement of the graft. When the tightening of the cord will be sufficient to ensure that the mobile sleeve can no longer slide on the cord, any advance of the mobile sleeve until complete fitting in the peripheral sleeve will necessarily be accompanied by a concomitant movement of forcing back the cord, i.e., taking place in the opposite direction of the tensioning movement of the graft. Such a movement can be overlooked when it takes place only at one end of the bone tunnels, the expansion of the system being able to then be corrected by the tensioning of the graft at the other end of the bone tunnels before its final locking. However, if, having attached one end of the graft, the same fixation system is applied at the other end of the graft, an irreparable loss of tension will inevitably be caused that can lead to a premature mechanical failure of the ligament reconstruction.

The publication of patent application US 2007/0162022 A1 proposes another fixation system intended to wedge a ligament by a tightening effect in the center of two half-pins introduced into a bone tunnel. As for the preceding invention, the implementation must necessarily take place at the exit of the bone tunnel with the known negative consequences in terms of elasticity. Furthermore, this system necessitates attaching the graft in advance to a half-anchor by means of threads of sutures. Still more than for the preceding system, the introduction of the pin into the bone recess inevitably causes an expelling effect of the graft that, being secured to the pin, is moved forcibly in the same direction as it, i.e., in the opposite direction of the movement for tensioning the graft. Finally, the tightening quality remains dependent upon the quality of the surrounding bone tissue, a soft bone leading to a less effective tightening effect than a hard bone. The variability of the quality of the fixation obtained by this system, moreover, has not escaped their authors who recommend an additional safety fixation by tying the graft-anchor suture threads to another stopping element fixed in the bone at the exit of the bone tunnel.

Utility certificate DE 20 205 017 919 U1 describes a device that makes it possible to wedge an artificial ligament in the center of a cylindrically-shaped tightening element that can be deformed radially when it is forced to penetrate into a peripheral sleeve using a screw. This system, however, necessarily requires the use of a tube-shaped artificial ligament, making it possible to introduce into its center a hooking tip that is essential to the functioning of the system.

This invention describes a wedging system that preferably addresses cords with a non-tubular solid section.

This invention makes it possible to avoid these two problems. It actually proposes a pin system with two components also but whose shape and characteristics make it possible to use it at any depth of the bone tunnel, preferably at a small distance from the graft, and, after adjustment of the ideal position, to obtain its locking without danger of pushing back and of relaxation of the cord to be attached.

Moreover, in the TLS system already mentioned, the elasticity of the bridge is neutralized after its introduction into the knee, during the placing of the TLS screw. This invention proposes applying to the band an additional change making it possible to have a cord structure that makes it possible to neutralize this elasticity before its introduction into the knee.

### DETAILED DESCRIPTION OF THE INVENTION

The invention consequently relates to a pin and a cord, derived from a band, suitable for a new system for fixation of a ligament graft.

During the factory production, after having been folded once on itself creating a free loop handle intended to suspend the graft, the two strands of the band are joined and secured to one another by an additional process transforming the flexible band with two free strands (as in the TLS system) into a tubular or threadlike structure of fine caliber, rigid or semi-rigid, making possible a fixation at a distance without significantly increasing the elasticity of the system. This tubular or threadlike structure comprising a loop handle at one end is hereinafter called a cord.

Three distinct processes for obtaining new cords according to an embodiment of the invention are described below and can be considered separately because they lead to fixation systems that are distinct within the limits of the individual inventive concept of the invention.
1. Stiffening by machining of the fibers. After formation of the flexible loop handle intended to suspend the graft, the fibers of each band strand can be joined and woven with one another to form a semi-rigid, elongated, cylindrical structure.
2. Stiffening of the bands by a heat-shrinkable sleeve. Certain materials (example: a crosslinked polyolefin) make possible the manufacture of sleeves whose diameter shrinks when they are subjected to a high temperature (80-90° C.). These materials are widely used in electricity for insulation and cladding of wires. There is a wide variety of heat-shrinkable sleeves of any size, with or without adhesive, of variable stiffness and of variable restricted capacity (from 2/1 to 4/1). A good number of these polymers are biocompatible and widely used in medicine. Certain materials are gradually resorbed over time and could also offer advantageous possibilities to this invention that in no case can be reduced to a single particular substance.
3. Stiffening by inclusion of the bands within a biocompatible polymer substance. Numerous biomaterials (polylactic acid, polyglycolic acid, etc.) are used in medicine (vascular surgery, dental surgery, bone and ligament surgery, etc.). The properties of these substances depend greatly on the mixtures of substances and on the proportions used, and this document does not have as its object to describe a particular chemical composition that is specially suited to this invention. Several of these biocompatible chemical substances could be suitable to create, for example by molding, a cylindrical structure that includes the two strands of bands, separate or pre-assembled by weaving, suitable for the fixation of the band thus modified. Ideally, so as to meet the requirements of the surgical technique, this coated structure should be stiff during axial mechanical stresses but relatively flexible to permit its positioning in the knee by arthroscopic surgical approaches.

If the flexibility of a continuous structure were insufficient to meet the requirements of the surgical technique, the bands can be coated in multiple and intermittent polymer segments by arranging intermediate band portions that are not included that obtain for the unit the desired flexibility.

The molding process potentially makes it possible to create on the outside surface of the polymer “sleeve” a raised feature that is especially suited to the processes for fixation by self-locking.

These latter two hybrid solutions (sleeve or inclusion) have multiple advantages:

A bridging element is obtained that is both very rigid and very resistant to axial pulling thanks to the core of the element composed of Dacron bands whose strength reaches 180 to 200 daN.

This strength is obtained for a very small size (cylindrical structure of 4-5 mm). Of course, the size of an element that is composed only of stiff material is generally larger to be able to compete with the tensile strength of the Dacron® bands (particularly at the suspension loop handle). The elasticity of the element is neutralized by engineering thanks to the addition of the shrinkable sleeve or by that of the polymer-coated wire cylinder.

The invention will be further understood on reading the following description taking into account the accompanying drawings, provided only by way of nonlimiting examples. The different structural elements of the invention and their arrangements are illustrated in these drawings in which:

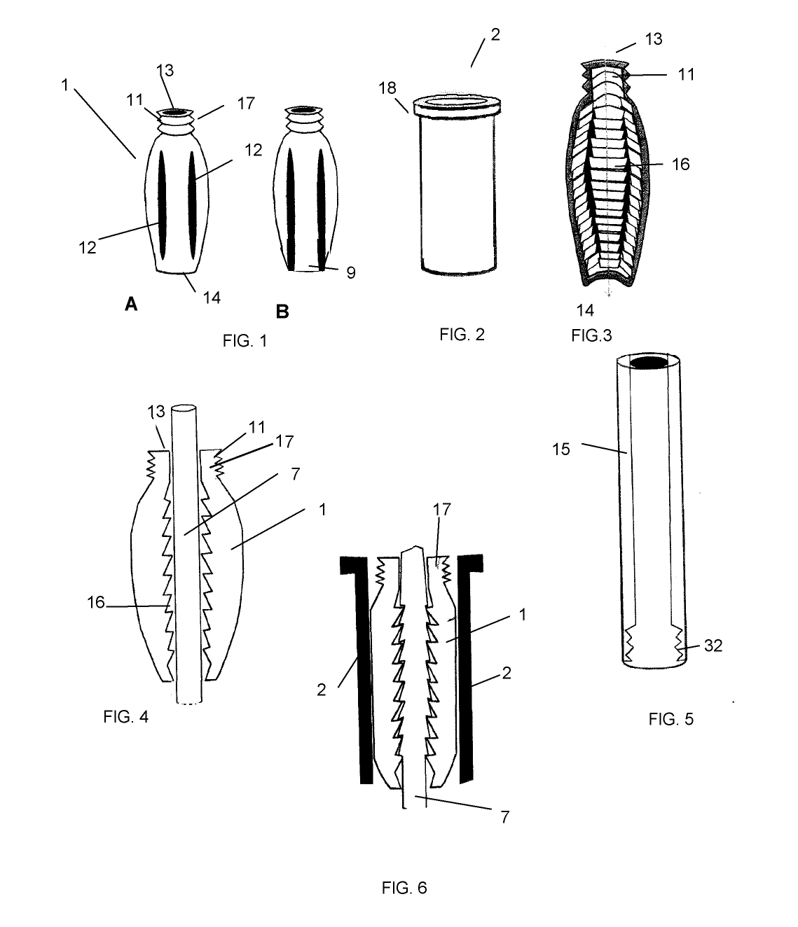

FIG. 1 represents a pin according to an embodiment of the invention,

FIG. 2 represents a compression cap-sleeve, to be applied on the pin,

FIG. 3 represents a cutaway view showing the inside of the pin,

FIG. 4 represents a view in section of the pin in open position, a cord sliding freely in the center of the pin,

FIG. 5 illustrates a pin holder,

FIG. 6 is a cutaway view showing the pin in closed position with the compression sleeve and the cord whose sliding is no longer possible,

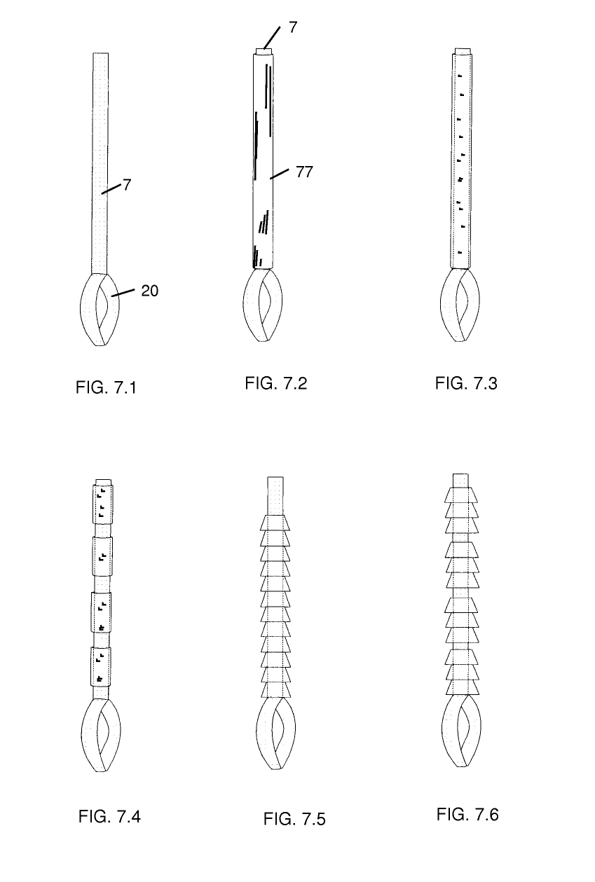

FIG. 7 illustrate various structure possibilities of the cord, according to various embodiments of the invention, either obtained by weaving 7.1, cladding 7.2, continuous inclusion 7.3, intermittent inclusion 7.4 with smooth surface, continuous inclusion with pyramid raised features 7.5, intermittent inclusion with pyramid raised features 7.6,

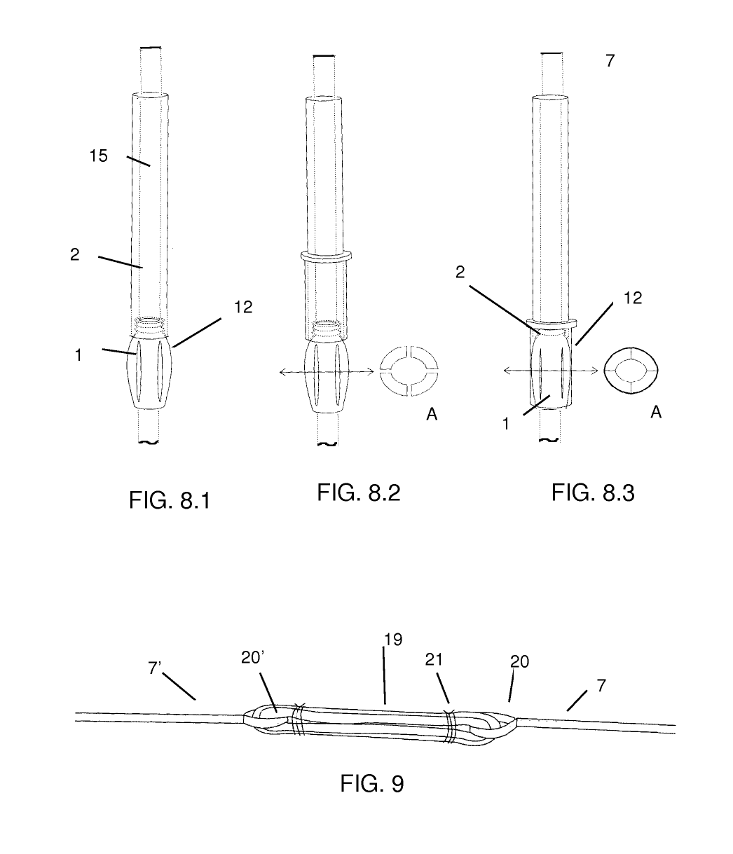

FIG. 8 illustrate the stages leading to the closing of the pin once placed in the bone (not shown),

FIG. 9 illustrates a graft according to the invention equipped with these two cords at the ends,

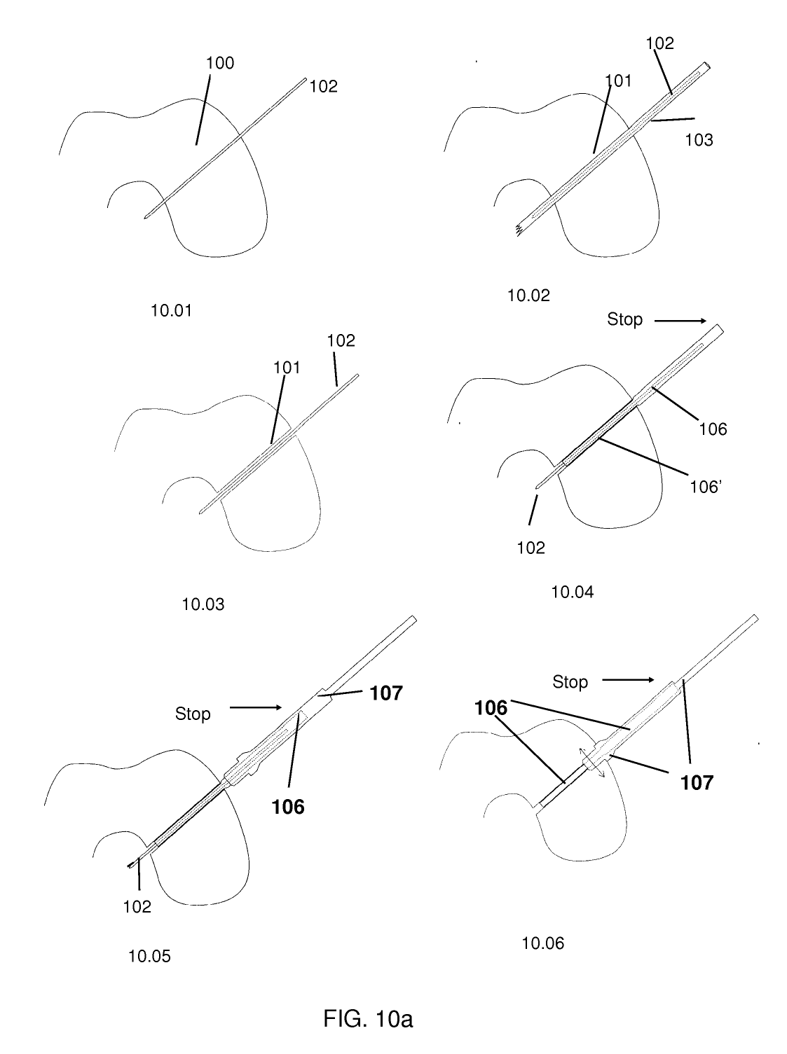

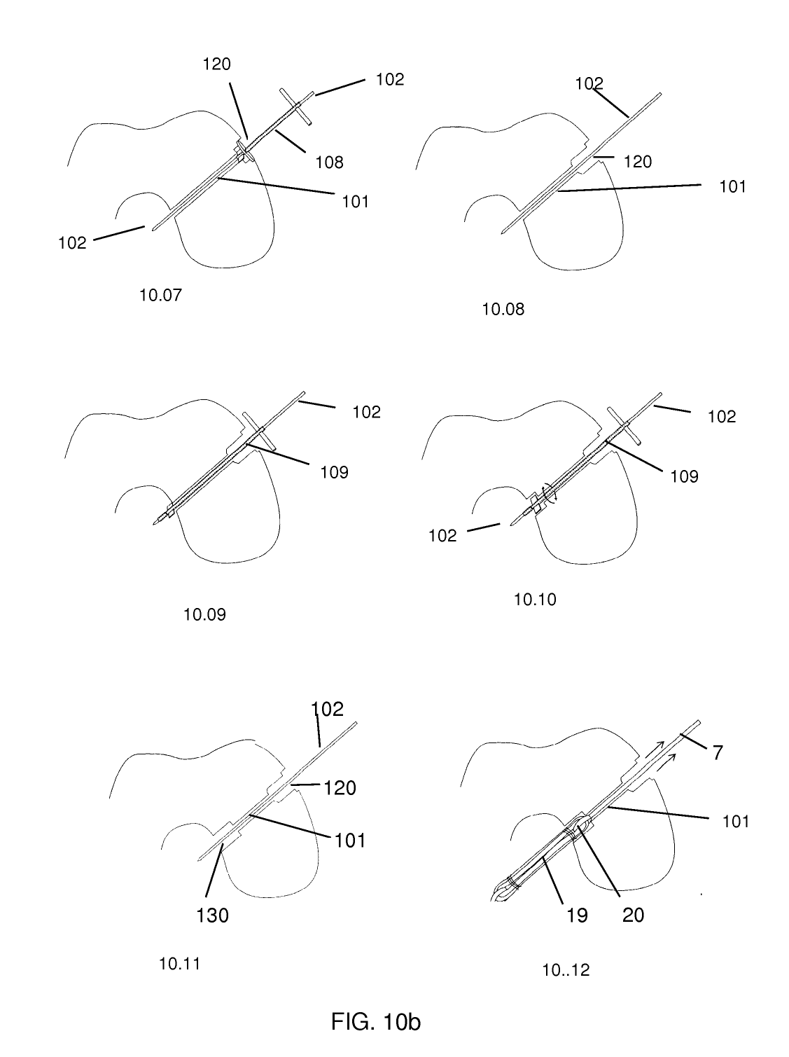

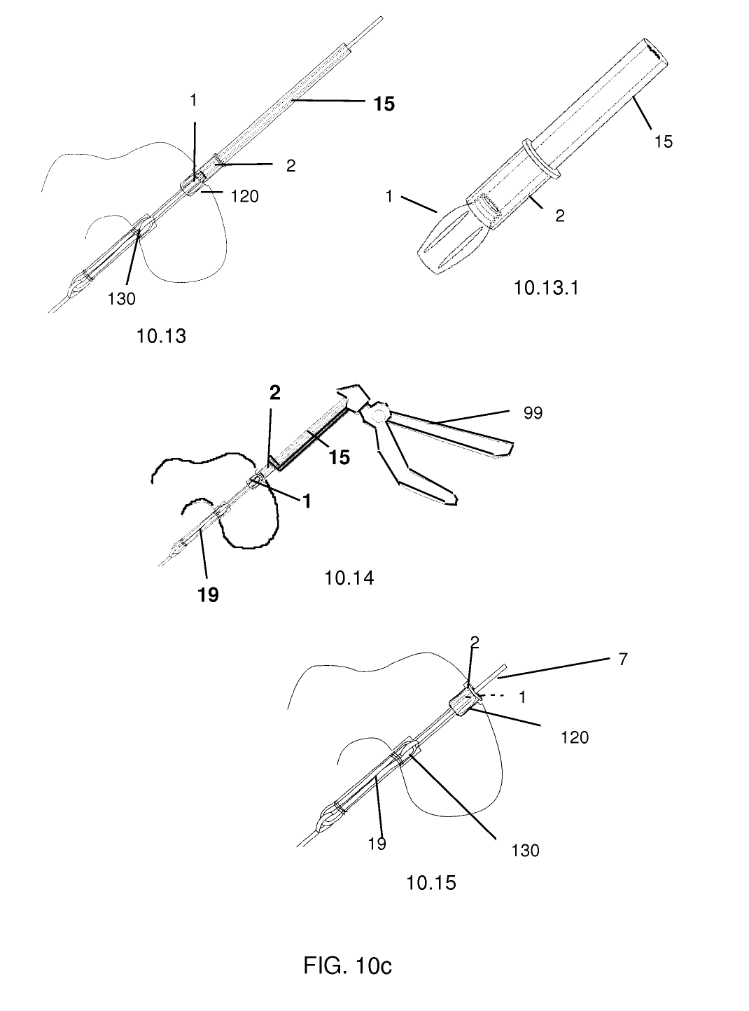

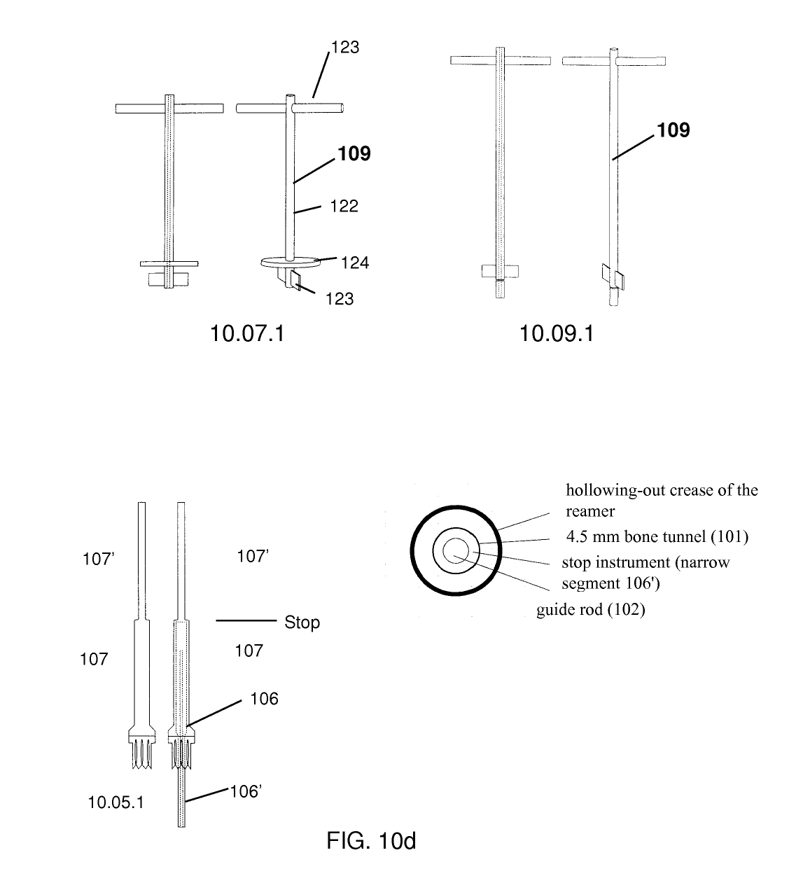

FIG. 10 explain diagrammatically the stages of the operating method for the placement of the graft as well as the additional tools necessary for the method,

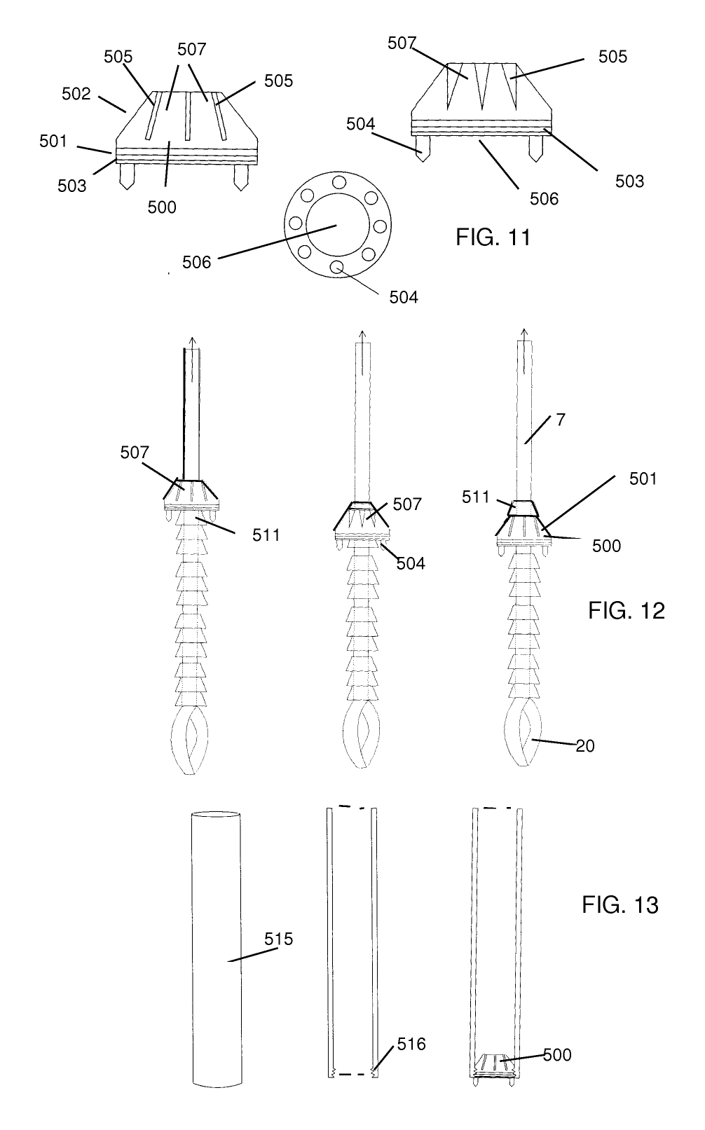

FIG. 11 illustrates another embodiment of the invention in which the pin is a self-locking pin,

FIG. 12 shows the functioning of this self-locking pin in conjunction with a cord according to the invention,

FIG. 13 illustrates the pin-holder tube with the self-locking pin.

The fixation of these traction elements (cords) can therefore be carried out in different ways but in particular according to two distinct methods, described successively below:

1. Fixation by Compression Pin

Referring to FIGS. 1 to 6, the fixation assured by a special pin system composed of two separate parts, the body of the pin 1 in the center of which the cord 7 of the fixation element freely slides, and the compression sleeve 2 intended to fit together with hard friction on the body of the pin is illustrated, calling for a deformation of the walls of the pin in the direction of its center, the place where the cord of the element passes. In contrast with the TLS screw that compresses the band outward, against the bone walls of the tunnel, the hollow cylindrical compression cap causes a compression of the cord inward, i.e., the central axis of the pin. It is seen right away that the resistance to pullout of the TLS fixation depends on the surrounding bone quality whereas the fixation of the single cord according to the invention depends on the “press-fit” effect obtained by the fitting of the cap on the body of the pin. Being dependent on the manufacturing characteristics of the pin, this effect can be standardized and is therefore reproducible from one case to the next regardless of the quality of the surrounding bone.

As already mentioned, an important property of the invention resides in the fact that during the locking of the cord by the pin, the wedging element remains fixed in relation to the cord, and it is the tightening cap that is moved. This mechanism makes it possible to avoid the unwanted expelling of the cord that inevitably occurs if the wedging element must be moved into the fixed peripheral pin to obtain the wedging. Thanks to this mechanism, this fixation can be used at both ends of the bone tunnel without causing expansion within the attached system, in contrast with most pins with two components where the peripheral element is fixed, and it is the wedging element that is moved in the direction opposite the tensioning movement of the system.

The body of the pin 1 can present itself in the form of an egg-shaped oblong element, pierced with a cylindrical cavity about 4 mm in diameter, which goes through the pin from one side to the other (from 13 to 14), in its center, along its major axis.

One of the ends 13 of the pin extends by a tubular appendage 17 that is 5 mm long and 6 to 7 mm in outside diameter, whose outer wall is equipped with a thread 11 intended to be screwed onto the pin-holder tube 15 at 32. The inside cavity of the pin goes through this appendage from one side to the other along its longitudinal axis and comes out on an opening that corresponds to the exit opening 13 for the cord. The other end of the pin also has an opening that corresponds to the entry opening 14 for the cord.

The outer wall of the pin is entirely smooth so as to facilitate the sliding of the compression cap, except for its proximal part, near its tubular appendage 17, which can have a raised feature in the shape of a groove, a rib or screw threads. An additional raised feature, but offset from the inside wall of the compression cap, will make it possible, after having fitted the two components by translation, to lock them, in relation to one another, by transmitting a partial rotation, for example a simple quarter- or half-turn, to the compression cap in relation to the wedging element. Thanks to this locking mechanism, a pull on the cord would not make it possible to dislocate the two components in the case where the recess for the ligament would communicate with the recess for the pin (for example in the case of small-size femurs calling for a very short bone tunnel). In these cases, the migration of the pin would be entirely blocked at the entrance of the bone tunnel by the annular element of the compression cap (and no longer by the bottom of the recess for the pin), provided that the dislocating of the pin from the compression cap had been neutralized by the above-described screwing mechanism. Other locking processes for the two components, such as a ratchet and pawl mechanism, could also be used.

The inside wall of the pin, in contrast, is provided with rows of sharp raised features 16 arranged in rings, superposed on one another, perpendicular to the major axis of the pin, lining the entire wall of the pin, from one end to the other. The section of these raised features presents, for example, a triangle shape whose base belongs to the wall of the pin and whose apex is inclined obliquely toward the opening for the exit of the cord. The cord slides easily in one direction, when it is moved in the direction of the exit opening of the pin, but its return is counteracted by the catching of the cord on the tip of the inside raised features 16 when it is drawn toward the entry opening of the pin. The wall of the pin is pierced with at least 4 longitudinal spindle-shaped slots 12 that are 7 to 8 mm long and 1 to 3 mm wide at the equator of the spindle. These slots, equidistant from one another, make it possible for the four solid segments of the pin to draw closer under the effect of a compression force applied perpendicular to the major axis of the pin. In the free state, the diameter of the central cavity of the pin therefore corresponds to the sum of the circumference portions of the 4 solid segments and of the 4 slots of the pin (see FIGS. 8.2). In case of compression of the pin along its transverse axis, the slots disappear and the solid segments draw close to one another until the diameter of the pin is reduced to the sum of the circumference portions of the 4 solid segments only (FIG. 8.3). The sharp top of the inside raised features of the pin then penetrates into the body of the cord completely neutralizing its sliding.

Referring to FIG. 2, the compression cap 2 is a hollow cylindrical element with a length identical to or slightly less than that of the body of the pin and whose inside diameter is less than the outside diameter of the body of the pin. It has at one end an annular segment 18 with a thickness (edge) of 2 to 3 mm whose outside diameter exceeds by 2 to 3 mm the outside diameter of the cylinder. This thickening is intended to press on the bone bordering the bone recess for receiving the pin, thus preventing it from penetrating too deeply during its introduction. The inside wall of the cylindrical segment of the cap is smooth except for its proximal part, near the annular segment, where a raised feature in the form of screw threads is found that is intended to lock the pin equipped with an additional raised feature after fitting by a screwing movement (¼ or ½ turn). FIG. 8 illustrate the mode of action of the pin.

FIG. 8.1 thus shows the body of the pin screwed onto the pin holder. FIG. 8.2 shows the same configuration but with the compression cap 2 overhanging the body of the pin 1 and ready to be pressed on. At this stage, the cord 7 slides freely in the body of the pin whose inside diameter is at its maximum dimension. FIG. 8.3 illustrates the appearance of the pin after pressing on the compression cap that reduces the inside diameter of the pin to its minimum dimension. The sharp raised features 16 of the wall counteract any sliding as illustrated by FIGS. 4 and 6.

The technique for using this pin for the fixation of an anterior cruciate ligament can be described as follows:

Preparation of the Graft (FIG. 9).

The semitendinosus is harvested in a standard way by means of a stripper. As in the TLS technique, the tendon is prepared by winding it on itself to form a short closed graft 19 having 4 strands making it possible to suspend it by means of a loop handle 20 of the fraction cord 7 placed at each end of the graft. In the TLS technique, the bands pass freely through each end of the graft and can therefore be introduced after making the graft.

In this technique, the loop handles 20 are closed and must therefore be put in place at the moment of making the graft. For the rest, as in the TLS technique, the tendon strands are fastened to one another by one to two suture points 21 placed at each end of the graft.

Preparation of the Tunnels 101 (See FIG. 10) in the Bone 100.

This is carried out in the same manner as the TLS technique by placing, from outside to inside, guide rods 102 whose end comes out in the center of the femoral and tibial intra-articular insertion point of the graft. The hollowing-out of the transbone tunnels is performed from outside to inside by means of hollow augers 103 that slide on the rod 102 guiding their path.

Preparation of the Recess for Receiving the Pin.

It is performed by means of a special cannulated reamer 107 (see FIG. 10d 10.05.1) that makes it possible to hollow-out the recess from outside to inside. Before the reaming, introduced onto the guide rod is a stop instrument 106 on which the reamer will come to stop after having made a recess to the desired standard dimension. This instrument, also illustrated in 10.05.2 (FIG. 10d), comprises two segments of different diameters continuous with one another, pierced with a cylindrical cavity making possible the sliding of the guide rod 102. The narrow segment 106′ has an outside diameter corresponding to or very slightly less than the diameter of the bone tunnel. When the tube is introduced from outside to inside, on the guide rod 102, the small diameter segment 106′ penetrates into the bone 100 until the wide segment of the instrument 106 stops at the entrance of the bone tunnel 101, its diameter being greater than that of the bone tunnel. FIG. 10d in 10.06 (cf. pin reaming) illustrates the appearance of the stop instrument stopped on the cortical bone at the widening of diameter of the instrument.

The reamer itself comprises a continuous reaming element with an elongated cylindrical segment that carries the reaming element and makes it possible to operate it. The reaming element has the shape of a hollow cylinder whose outside, cutting raised features are especially suited to bone reaming. The height of the cylinder corresponds to the desired depth of hollowing-out (15 to 20 mm). It has an outside diameter of 10 mm that corresponds to the desired drilling site diameter almost over its entire height, except for its proximal part, where this diameter widens 3-4 mm over a height of about 2 mm. The inside diameter of the central cavity of the cylinder corresponds to or is slightly greater than the wide segment of the stop instrument.

The elongated cylindrical segment that carries the reaming element has a wide, hollow segment that is continuous with a solid cylindrical segment 107′ whose proximal end can be equipped with several flat sides intended to optimize its hold by the chuck of the motor. The inside diameter of the hollow segment corresponds to or is very slightly greater than the outside diameter of the wide segment of the stop instrument, so that it can slide on it. The total length of the hollow segment of the reamer corresponds to the length of the wide segment of the stop instrument increased by the length of the reaming element. FIG. 10 clearly show that when the bottom of the cavity of the reamer stops on the outside end of the stop instrument, the reaming element has penetrated the bone to the desired, still identical, depth depending on the sides of the instrument. The measurements that are cited here by way of example in no way constitute a specific characteristic of the instruments described. The cross-section passing through the reaming zone clearly shows that after passage of the reamer, a cylindrical-shaped bone segment will persist in the center of which is found the bone tunnel containing the stop instrument (narrow segment) with the guide rod in its center. It is on this bone cylinder that the wide segment of the stop instrument rests, determining the penetration depth of the reamer. After passage of the reamer, it will be necessary to resect this bone cylinder and completely clean the recess 120 by means of a small hand reamer 108 illustrated in FIG. 10d in 10.07. It is composed of a hollow tube 122, able to slide on the guide rod 102 and equipped with a handle 123 at its proximal end.

Its distal end has two cutting blades 123 joined together at the end of the tube, perpendicular to its major axis. The size of the blades corresponds to the desired inside diameter of the recess (10 mm). A circular-shaped part 124 with a diameter equal to the widened proximal diameter of the reamer is fastened perpendicular to the tubular axis of the instrument, at a distance corresponding exactly to the desired depth of the recess. A few circular movements of this instrument will make it possible to eliminate all of the bone debris from the recess without the least risk of excessive penetration thanks to the circular stop 124 coming to rest on the bone raised feature created by the reamer 107 in 10.06. In 10.07 and 10.08 (FIG. 10b), the production and the appearance of the recess continuous with the bone tunnel after reaming is shown. In 10.11.1, the different diameters of the recesses thus created are illustrated.

Preparation of the Recess 130 for Receiving the Graft.

The technique for making the reception recesses for the ligament graft is strictly identical to that described in the TLS technique. It is carried out by means of hollow augers 109 equipped with cutting blades whose size corresponds to the diameter of the graft. They are introduced into the knee with a hammer, from outside to inside, until they reach into the articular cavity. A combined traction and rotational movement will then cause the retrograde hollowing-out of the recess (see FIG. 10.09).

Introduction of the Graft

The graft is introduced into the knee first by anterointernal means. The cord 7 with the femoral traction loop handle 20 is passed into the femoral tunnel 101 by its intra-articular opening and is recovered at the external face of the femur. The traction on this cord 7 causes the penetration of the graft 19 into the femoral recess 130 until it stops on the bottom of this recess.

Locking of the Graft

The pin 1 equipped with the compression cap 2 in free position is mounted on the carrying tube 15. The pin and its tube are then slipped on the cord 7, and while maintaining tension on the cord, the pin is pushed until it stops on the bottom of its recess 120. Then, a pushing instrument 99 is used, one arm of which is fastened on the pin-holder tube 15 and the other arm of which, mobile, makes it possible to exert a strong pressure, directed toward the pin, on the compression cap, which penetrates into the recess by compressing the pin and by blocking any movement of the central cord.

2. Fixation by Self-Locking Pin

Referring to FIGS. 11 to 13, a variant of the invention is illustrated consisting of a self-locking pin in the form of a hollow element having a cylindrical segment 501, extended by a conical section 502 having a truncated top. The cylindrical part has a constant diameter over a height of about 3-4 mm. The outside wall of this segment possesses a screw thread 503 making it possible to fasten a pin holder there. The inside wall of this segment, also cylindrical, has a diameter corresponding to or slightly greater than the largest diameter of the pyramidal or conical elements that are present along the cord, in such a way that they can easily penetrate into this first segment.

According to a currently preferred embodiment, the base of this segment is equipped with an anti-rotation device in the form of small tips 504 arranged around the entry opening 506 of the cord. If the thickness of the wall is insufficient to create these rough edges there, a toothed raised feature can also be created over the periphery of the entry opening of the cord. The role of these devices is to facilitate the unscrewing maneuver of the pin holder after implantation of the graft by counteracting an unwanted rotational movement of the pin transmitted by the pin holder.

The inside and outside diameter of the conical segment is equal to that of the cylinder at its junction with it, then is rapidly reduced in the direction of the truncated top of this segment, to attain a dimension that is clearly less than that of the fragments of the cord. This conical segment is equipped with several longitudinal slots 505 that reach the top and that divide the cone into sections 507 that meet at their base and that are separated at their top. A cord equipped with pyramidal or conical fragments 76, drawn from the base of the pin toward its top, therefore forces the sections 507 to separate from one another until a fragment has gone through the exit opening. At this time, the sections draw closer again by simple elasticity, counteracting any possibility of return movement.

The placement technique is carried out very simply, similar to the technique described for the compression pin.

It will necessitate a small bone reaming to create the receiving recess for the pin, by means of instruments suited to the final shape of the implant. It also necessitates the use of a special instrument that makes it possible to exert a pull on the cord while pushing on the pin-holder tube 515.

FIG. 11 illustrates a self-locking pin 500 in closed position, in open position and seen from its base.

FIG. 12 illustrates the introduction of a cord 76 through the self-locking pin 500. A first trapezoidal unit 511 is passed through this pin 500 (previously introduced into its recess, not shown). The traction on the cord 7 (while keeping the pin in place thanks to the pin-holder tube 515 illustrated in FIG. 13) leads to the penetration of the trapezoidal unit that opens the wings 507 of the pin. Finally, the unit has gone through the small opening of the pin, and the wings are closed again, preventing any possible return of the cord. According to the desired adjustment, the operation can be repeated with the trapezoidal elements that follow.

FIG. 13 illustrates a pin-holder tube 515 in elevation, then in section with the screw thread 516, and then together with a self-locking pin 500.

1-27. (canceled)

28. Pin (1) suited for a system for fixation of a ligament graft consisting of an oblong body, for example egg-shaped, pierced with a cylindrical cavity able to receive a cord, which goes through the pin from one side to the other, in its center and along its major axis, while passing through an entry opening and an exit opening for said cord, said body being able to be compressed toward said axis under the effect of an outside push perpendicular to said axis and thus to immobilize said cord.

29. Pin according to claim 28, whose wall is pierced with one or more grooved longitudinal slots.

30. Pin according to claim 28, in which the exit opening is extended by a tubular appendage whose outside wall is equipped with a thread able to be screwed onto a pin holder.

31. Pin according to claim 28, in which the outside wall is entirely smooth.

32. Pin according to claim 28 in which the inside wall is provided with rows of raised features in which the section of the raised features has a shape of a triangle whose base belongs to the wall of the pin and whose apex is inclined obliquely toward the exit opening for the cord.

33. Pin according to claim 28 in which the cylindrical cavity is 2 to 6 mm.

34. Pin according to claim 28 in which at the outside wall, preferably adjacent to the exit opening, a raised feature is provided, for example a partial screw thread able by rotation to fit into a complementary raised feature of a second component appearing in the form of a compression cap, thus causing an interlocking of said pin with said cap.

35. Cord for a system for fixation of a ligament graft obtained by folding a band or thread once on itself creating a free loop handle intended to suspend said graft, the two strands of said band being joined and interlocked with one another by an additional process changing said flexible band with two free strands into a rigid or semi-rigid tubular threadlike structure, entirely or by sections, equipped with a flexible loop handle at one end.

36. Cord according to claim 35, in which the stiffening is obtained by machining the fibers.

37. Cord according to claim 36, in which the stiffening is obtained by application of a thermosettable sleeve and/or by inclusion of a hardenable polymer substance.

38. Ligament graft with fixation-bridging means comprising a tendon or tendon element folded and sutured in a closed loop at at least one end, in which the fixation means is a bridging cord in the form of a thread or band that is folded to form a flexible loop handle enclosing said loop, characterized in that the two free strands of the thread or band are interlocked and/or stiffened in a single, rigid or semi-rigid threadlike structure.

39. Graft according to claim 38 in which the stiffening is obtained by machining the fibers of the thread and/or by heat-shrinking and/or application of a thermosettable sleeve and/or by inclusion of a hardenable polymer substance.

40. Graft according to claim 38, in which the stiffening is obtained by inclusion of a solidifiable biocompatible polymer substance within the bands or threads whose strands have been previously gathered together, potentially pre-assembled by weaving and/or braiding.
